(19) United States
(12) Patent Application Publication
(10) Pub. No.: US 2010/0260736 A1
(43) Pub. Date: Oct. 14, 2010

(54) EGR1 MODULATORS FOR THE TREATMENT OF ALOPECIA

(75) Inventor: Sandrine RETHORE, Valbonne (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/784,672

(22) Filed: May 21, 2010

Related U.S. Application Data
(63) Continuation of application No. PCT/FR08/52131, filed on Nov. 26, 2008.

(30) Foreign Application Priority Data
Nov. 26, 2007 (FR) 0759323

(51) Int. Cl. A61K 38/43; A61P 17/14; C12Q 1/02; C12Q 1/68; G01N 33/53

(52) U.S. Cl. 424/94.1; 435/29; 435/6; 435/7.92

(57) ABSTRACT

An in vitro method of screening of candidate compounds for the preventive or curative treatment of alopecia includes determination of the capacity of a compound to modulate the expression or activity of the Early Growth Response 1 transcription factor (EGR1), as well as the use of modulators of the expression or activity of this transcription factor for the treatment of alopecia; such method also includes in vitro diagnosis or prognosis of this pathology.

### CROSS-REFERENCE TO PRIORITY/PCT APPLICATIONS

This application claims priority under 35 U.S.C. §119 of FR 0759323, filed Nov. 26, 2007, and is a continuation of PCT/FR 2008/052131, filed Nov. 26, 2008 and designating the United States (published in the French language on Jun. 11, 2009 as WO 2009/071841 A2; the title and abstract were also published in English), each hereby expressly incorporated by reference in its entirety and each assigned to the assignee hereof.

### BACKGROUND OF THE INVENTION

1. Technical Field of the Invention

The present invention relates to the identification and administration of compounds that are modulators of the Early Growth Response 1 transcription factor (EGR1), for the treatment of alopecia. This invention also relates to methods of in vitro diagnosis or in vitro prognosis of this pathology.

2. Description of Background and/or Related and/or Prior Art

In humans, hair growth is cyclic and comprises three successive phases: the anagen phase, the catagen phase and the telogen phase. Each follicle of the hair is thus constantly being renewed, cyclically and independently of the adjacent follicles (Kligman 1959, Montagna and Parakkal, 1974). The anagen phase or growth phase, over the course of which the individual hairs increase in length, lasts several years. This phase recapitulates the morphogenesis of the hair and is divided into 7 different stages (anagen I to anagen VII) (Muller-Rover et al., 2001). For simplicity, the anagen phase is generally reduced to three stages, each comprising several substages: early for stages I-III, middle anagen for stages IV to V and late anagen for stages VI and VII.

The catagen phase, which follows the anagen phase, is very short and only lasts a few weeks. This phase is divided into 8 different stages (catagen I to catagen VIII) (Muller-Rover et al., 2001). During this phase, the hair undergoes involution, the follicle atrophies and its dermal implantation appears to be higher and higher. The telogen phase, which lasts some months, corresponds to a resting period of the follicle in which the hair is finally shed. After this resting phase a new follicle is regenerated, in situ, and the cycle begins again. (Montagna and Parakkal, 1974).

At any given moment, not all hairs are in the same phase at the same time. Thus, of the 150,000 hairs making up a head of hair, only about 10% of them are at rest and will therefore be replaced in a few months according to the biological clock of each hair (Montagna, 1974).

In mice and other mammals with fur, the hair follicles similarly possess a cycle of renewal comprising the three phases—anagen, catagen and telogen, divided into various stages. In contrast, the hair cycles of young animals are often “synchronized”, i.e., in the same phase of the cycle at the same moment within one and the same anatomic region (Muller-Rover et al., 2001).

Natural hair loss is a physiological phenomenon that takes place constantly and can be estimated, on average, at a few hundred hairs per day for a normal physiological condition. However, the hair cycle can become uncontrolled and hair loss can accelerate, leading to a temporary or permanent hair loss called alopecia. Alopecia can have various causes.

There are various types of alopecia, the main forms being:
hereditary androgenetic alopecia is the commonest: it is manifested by a decrease in hair volume, or even baldness, and affects 70% of men;
acute alopecia: this may be associated with treatment by chemotherapy, stress, severe malnutrition, iron deficiency, hormonal disorders, AIDS, acute irradiation;
http://fr.wikipedia.org/wiki/Syndrome d%27irradiation aig%C3%BCe alopecia areata, which seems to be of auto-immune origin (mechanism of cellular mediation) and is characterized by “patches” of varying size and in one or more places. This form of alopecia can affect the whole scalp and is then called alopecia totalis, and sometimes the whole body, then being called alopecia universalis, there in this case being not a single bristle or hair left on the entire body.

In all these three cases, hair loss is directly related to the hair cycle, as the follicle no longer enters the anagen phase, or the anagen phase is not maintained, which means that the follicle no longer produces a hair shaft and therefore no more hair. To combat alopecia it is therefore necessary to start the hair cycle again by activating the anagen phase.

Accordingly, for many years now, in the cosmetic or pharmaceutical industry, compositions have been sought that are able to eliminate or reduce alopecia, and notably to induce or stimulate start-up of the anagen phase or hair growth.

### SUMMARY OF THE INVENTION

It has now been determined that the gene coding for Early Growth Response 1 is expressed specifically in the keratinocytes of the hair follicle, and that its expression is induced at the moment of starting anagen, in vivo, in a model of induction of starting of anagen by gonadectomy. The present invention targets this gene or its expression product, to prevent or improve the manifestations of alopecia.

By alopecia is meant all forms of alopecia, namely in particular androgenetic alopecia, acute alopecia or alopecia areata.

EGR1:

The Early Growth Response 1 (or “EGR1”) gene codes for a zinc-finger protein of type C2H2 which is a member of the EGR family.

According to the present invention, the term “EGR1 gene” or “EGR1 nucleic acid” denotes the gene or the nucleic acid sequence that codes for the EGR1 protein. Although the target sought is preferably the human gene or its expression product, the present invention can also make use of cells expressing the Early Growth Response 1 transcription factor, by genomic integration or transient expression of an exogenous nucleic acid coding for the transcription factor.

The human nucleic acid sequence (SEQ ID No.1) and the human protein sequence (SEQ ID No.2) of the EGR1 transcription factor are reproduced in the appendix.

It is a nuclear protein that functions as a transcription factor, modulating genes involved in differentiation and mitogenesis. EGR1 is known to be expressed and to play an important role during tooth morphogenesis (Karavanova, 1992). A large number of genes and signaling pathways, present during morphogenesis of the teeth, are implicated in the hair cycle and in particular at the moment of starting of anagen. For example, the BMP pathway controls the start-up of development of the teeth and the start of the growth phase of the adult hair follicle (Botchkarev and Sharov, 2004). Specific expression of the EGR1 transcription factor in the keratinocytes of the hair and its induction during the start of anagen suggests that it plays an important role in the homeostasis of the hair follicle.

Diagnostic Applications:

One aspect of the invention features an in vitro method of diagnosis or of monitoring the development of alopecia in a subject, comprising comparison of the expression or activity of the Early Growth Response 1 protein (EGR1), expression of its gene or of the activity of at least one of its promoters, in a subject's biological sample, relative to a control subject.

The expression of the protein can be determined by an assay of said EGR1 protein by an immunohistochemical test or immunoassay, for example by ELISA assay. Another method, notably for measuring the expression of the gene, is to measure the amount of corresponding mRNA, by any method as described above. An assay of the activity of the EGR1 transcription factor can also be envisaged.

Within the scope of diagnosis, the “control” subject is a “healthy” subject.

Within the scope of monitoring the development of alopecia, the “control subject” refers to the same subject at a different time, which preferably corresponds to the start of the treatment (T₀). By measuring the difference in expression or activity of the EGR1 protein, expression of its gene or of the activity of at least one of its promoters, it is notably possible to monitor the efficacy of a treatment, notably a treatment with a modulator of the EGR1 transcription factor, as envisaged above, or by some other treatment against alopecia. This monitoring can comfort the patient as to the justification, or the need, to continue said treatment.

Another aspect of the present invention features an in vitro method of determination of a subject's likelihood of developing alopecia, comprising comparison of the expression or activity of the Early Growth Response 1 protein (EGR1), expression of its gene or of the activity of at least one of its promoters, in a biological sample from a subject relative to a control subject.

Once again, expression of the protein can be determined by an assay of the EGR1 protein, by an immunohistochemical test or immunoassay, for example by ELISA assay. Another method, notably for measuring the expression of the gene, is to measure the amount of corresponding mRNA by any method as described above. Assay of the activity of the EGR1 transcription factor can also be envisaged.

The subject tested is in this case an asymptomatic subject, not displaying any hair disorder associated with alopecia. The “control” subject, in this method, means a “healthy” subject or reference population. Detection of this susceptibility makes it possible to start preventive treatment and/or increased monitoring for the signs associated with alopecia.

In these methods of in vitro diagnosis or prognosis, the biological sample tested can be any sample of biological fluid or a sample from a biopsy. Preferably the sample can, however, be a preparation of skin cells, obtained for example by epilation of hair or biopsy.

Methods of Screening:

Another aspect of the invention is an in vitro method of screening of candidate compounds for the preventive and/or curative treatment of alopecia, comprising the determination of the capacity of a compound to modulate the expression or activity of the Early Growth Response 1 transcription factor (EGR1) or the expression of its gene or the activity of at least one of its promoters, said modulation indicating the usefulness of the compound for the preventive or curative treatment of alopecia. The method therefore makes it possible to select compounds capable of modulating the expression or activity of the EGR1 transcription factor, or the expression of its gene, or the activity of at least one of its promoters.

### DETAILED DESCRIPTION OF BEST MODE AND SPECIFIC/PREFERRED EMBODIMENTS OF THE INVENTION

More particularly, the present invention features an in vitro method of screening of candidate compounds for the preventive and/or curative treatment of alopecia, comprising the following stages:
a. preparation of at least two biological samples or reaction mixtures;
b. bringing one of the samples or reaction mixtures into contact with one or more of the compounds to be tested;
c. measurement of the expression or of the activity of the EGR1 protein, expression of its gene or of the activity of at least one of its promoters, in the biological samples or reaction mixtures;
d. selection of the compounds for which modulation of the expression or activity of the EGR1 protein, expression of its gene or of the activity of at least one of its promoters, is measured in the sample or the mixture treated in b), relative to the untreated sample or mixture.

“Modulation” means any effect on the level of expression or of activity of the EGR1 transcription factor, expression of its gene or of the activity of at least one of its promoters, namely optionally inhibition, but preferably stimulation, partial or complete.

Thus, the compounds tested in stage d) above preferably induce the expression or activity of the EGR1 protein, the expression of its gene or the activity of at least one of its promoters.

Throughout the present text, unless specified otherwise,
“expression of a protein” means the amount of said protein;
“Activity of a protein” means its biological activity;
“Activity of a promoter” means the capacity of said promoter to trigger the transcription of the DNA sequence encoded downstream of this promoter (and therefore indirectly the synthesis of the corresponding protein).

The compounds tested can be of any type. They can be of natural origin or can have been produced by chemical synthesis. It can comprise a bank of structurally defined chemical compounds, of compounds or of substances that have not been characterized, or a mixture of compounds.

Various techniques can be employed for testing these compounds and identifying the compounds of therapeutic interest, modulators of the expression or activity of the EGR1 transcription factor.

According to a first embodiment, the biological samples are cells transfected with a reporter gene bound operatively to all or part of the promoter of the EGR1 gene, and stage c) described above entails measuring the expression of said reporter gene.

The reporter gene can notably code for an enzyme which, in the presence of a given substrate, leads to the formation of colored products, such as CAT (chloramphenicol acetyltransferase), GAL (beta galactosidase), or GUS (beta glucuronidase). It can also be the gene of luciferase or of GFP (Green Fluorescent Protein). Assay of the protein encoded by the reporter gene, or of its activity, is carried out conventionally, by colorimetric, fluorometric, or chemiluminescence techniques, among others.

According to a second embodiment, the biological samples are cells expressing the gene coding for the EGR1 transcription factor, and stage c) described above entails measuring the expression of said gene.

The cell used here can be of any type. It can be a cell expressing the EGR1 gene endogenously, for example a liver cell, a prostate cell, or better still a skin cell, keratinocytes of the hair follicle or fibroblasts of dermal papillae. It is also possible to use organs of human or animal origin, for example hair, or hair follicles of vibrissae.

It can also be a cell transformed by a heterologous nucleic acid, coding for the EGR1 transcription factor, preferably human or mammalian.

A great variety of systems of host cells can be used, for example the Cos-7, CHO, BHK, 3T3, HEK293 cells. The nucleic acid can be transfected stably or transiently, by any method known to one skilled in the art, for example by calcium phosphate, DEAE-dextran, liposome, virus, electroporation, or microinjection.

In these methods, the expression of the EGR1 gene can be determined by measuring the rate of transcription of said gene, or its rate of translation.

“Rate of transcription” of a gene means the amount of corresponding mRNA produced. “Rate of translation” of a gene means the amount of corresponding protein produced.

One skilled in the art is familiar with the techniques for quantitative or semi-quantitative detection of the mRNA of a gene of interest. Techniques based on hybridization of the mRNA with specific nucleotide probes are the more usual (Northern Blot, RT-PCR, protection with Rnase). It can be advantageous to use detection markers, such as fluorescent, radioactive, enzymatic agents or other ligands (for example, avidin/biotin).

In particular, the expression of the gene can be measured by real-time PCR or by RNase protection. “RNase protection” means the detection of a known mRNA among the RNA-poly(A) of a tissue, which can be carried out by means of specific hybridization with a labeled probe. The probe is a labeled complementary RNA (for example radioactive or enzymatic) of the messenger to be found. It can be constructed from a known mRNA whose cDNA, after RT-PCR, has been cloned in a phage. RNA-poly(A) of the tissue where the sequence is to be found is incubated with this probe in conditions of slow hybridization in liquid medium. There is formation of RNA:RNA hybrids between the mRNA being sought and the antisense probe. The hybridized medium is then incubated with a mixture of specific ribonucleases of the single-stranded RNA, in such a way that only the hybrids formed with the probe can withstand this digestion. The digestion product is then deproteinized and purified again, before being analyzed by electrophoresis. The labeled RNA hybrids are detected for example by autoradiography or chemiluminescence.

The rate of translation of the gene is evaluated for example by immunoassay of the product of said gene. The antibodies used for this purpose can be of polyclonal or monoclonal type. Their production is based on conventional techniques. An anti-Early Growth Response 1 polyclonal antibody can be obtained inter alia by immunization of an animal such as a rabbit or a mouse, by means of the complete protein. The antiserum obtained is then extracted according to methods known per se by one skilled in the art. A monoclonal antibody can be obtained inter alia by the classical method of Köhler and Milstein (Nature (London), 256: 495-497 (1975)). Other methods of preparation of monoclonal antibodies are also known. Monoclonal antibodies can for example be produced by expression of a nucleic acid cloned from a hybridoma. Antibodies can also be produced by the phage display technique, by introducing antibody cDNA into vectors, which are typically filamentous phages that display V-gene banks on the surface of the phage (for example fUSE5 for E. coli).

Immunoassay can be carried out in solid phase or in homogeneous phase; once or twice; in sandwich mode or in competitive mode, as non-limiting examples. According to a preferred embodiment, the capture antibody is immobilized on a solid phase. As non-limiting examples of solid phase, it is possible to use microplates, in particular polystyrene microplates, or particles or solid beads, or paramagnetic beads.

ELISA assays, immunoassays, or any other detection technique can be employed for revealing the presence of the antigen-antibody complexes that have formed.

Characterization of the antigen/antibody complexes, and more generally of the isolated or purified proteins but also of recombinant proteins (obtained in vitro and in vivo) can be performed by mass spectrometry analysis. This identification is made possible by analyzing (determining the mass) of the peptides generated by enzymatic hydrolysis of the proteins (generally trypsin). In general, the proteins are isolated by methods known to one skilled in the art, prior to enzymatic digestion. Analysis of the peptides (in the form of hydrolysate) is performed by separating the peptides by HPLC (nano-HPLC) based on their physicochemical properties (reversed-phase). The mass of the peptides thus separated is determined by ionization of the peptides, either by direct coupling to the mass spectrometer (electrospray mode ESI) or after deposition and crystallization in the presence of a matrix known by one skilled in the art (analysis in MALDI mode). The proteins are then identified using suitable software (for example Mascot).

The EGR1 transcription factor can be produced by usual techniques using Cos-7, CHO, BHK, 3T3, HEK293 cells. It can also be produced by means of microorganisms such as bacteria (for example E. coli or B. subtilis), yeasts (for example Saccharomyces, Pichia) or insect cells, such as Sf9 or Sf21.

Transcription Factor Modulators

This invention also features the use of a modulator of the EGR1 transcription factor that can be obtained according to one of the methods described above for the preparation of a medicinal product intended for the preventive and/or curative treatment of alopecia.

Thus, a method of preventive and/or curative treatment of alopecia is described here, said method comprising the administration of a therapeutically effective amount of a modulator of the EGR1 transcription factor to a patient needing said treatment.

Preferably, said modulators are activators (or inducers) of the EGR1 transcription factor.

The present invention comprises the use of compounds that are inducers of the EGR1 transcription factor, such as those identified by the method of screening described above, for the preventive and/or curative treatment of alopecia.

The modulator compounds are formulated within pharmaceutical compositions, together with a pharmaceutically acceptable excipient. These compositions can be administered, whether regime or regimen, for example by the enteral, parenteral, or topical route. Preferably, the pharmaceutical composition is applied topically. For oral administration, the pharmaceutical composition can be in the form of tablets, capsules, sugar-coated pills, syrups, suspensions, solutions, powders, granules, emulsions, suspensions of microspheres or nanospheres or lipid or polymer vesicles providing controlled release. For parenteral administration, the pharmaceutical composition can be in the form of solutions or suspensions for infusion or for injection.

For topical application, the pharmaceutical composition is more particularly useful for the treatment of the skin, mucosae and scalp and can be in the form of unguents, creams, milks, ointments, powders, impregnated tampons, solutions, gels, sprays, lotions or suspensions. It can also be in the form of suspensions of microspheres or nanospheres or lipid or polymer vesicles or polymer patches or hydrogels providing controlled release. This composition for topical application can be in anhydrous form, in aqueous form or in the form of an emulsion. In a preferred variant, the pharmaceutical composition is in the form of a gel, a cream or a lotion.

The composition can have a content of modulator of Early Growth Response 1 transcription factor in the range from 0.001 to 10 wt. %, notably from 0.01 to 5 wt. % relative to the total weight of the composition.

The pharmaceutical composition can also contain inert additives or combinations of said additives, such as:
wetting agents;
flavor improving agents;
preservatives such as esters of parahydroxybenzoic acid;
stabilizers;
moisture regulators;
pH regulators;
osmotic pressure modifiers;
emulsifiers;
UV-A and UV-B filters;
and antioxidants, such as alpha-tocopherol, butyl hydroxyanisole or butyl hydroxytoluene, Super Oxide Dismutase, Ubiquinol or certain chelating agents of metals.

### BRIEF DESCRIPTION OF THE DRAWINGS

FIG. 1 illustrates induction of transition to anagen by ovariectomy. Female mice, whose hair follicles on the dorsal region were in telogen on Day 0, were ovariectomized or not (control) on Day 1 of the study. A skin sample was taken from the dorsal region of the mice on Days 0, 6 and 8 of the study. FIG. 1A shows a histological section of skin from the dorsal region of a mouse on Day 0 of the study. FIG. 1B is a photograph of a histological section of skin from the dorsal region of a mouse ovariectomized on Day 7 of the study. FIG. 1C shows a histological section of skin from the dorsal region of a mouse ovariectomized on Day 8 of the study. FIG. 1D shows a histological section of skin from the dorsal region of a control mouse on Day 8 of the study. Histological examination clearly shows that ovariectomy induced the transition to anagen (FIG. 1C).

FIG. 2 is a Table 1 which shows the modulation of the level of expression of the EGR1 transcription factor, expressed relative to Day 0 of the study, in the skin from the dorsal region of mice ovariectomized on Day 8 of the study and in the skin from the dorsal region of a control mouse (skin in telogen phase) on Day 8 of the study, using Affymetrix chip technology. Female mice, whose hair follicles on the dorsal region were in telogen on Day 0, were ovariectomized on Day 1 of the study. Non-ovariectomized mice were kept as a control group. A skin sample was taken from the dorsal region of the mice on Days 0 and 8 of the study. The RNAs were isolated and the expression of the genes was analyzed using Affymetrix chip technology.

FIG. 3 is a histogram representing the modulation of the EGR1 transcription factor, in the skin from the dorsal region of female mice expressed relative to Day 0 of the study, at the start of anagen induced by ovariectomy. Female mice, whose hair follicles on the dorsal region were in telogen on Day 0, were ovariectomized or not (control) on Day 1 of the study. A skin sample was taken from the dorsal region of the mice on Days 0, 1, 2, 4, 6 and 8 of the study. The RNAs were isolated and the expression of the genes was analyzed by means of Affymetrix chip technology. The analysis of gene expression clearly shows that the EGR1 gene is induced in the animals entering anagen.

FIG. 4 shows the expression, by hybridization in situ, of the EGR1 transcription factor in hair follicles at the start of anagen in the skin from the dorsal region of mice. FIG. 4A is a photograph of the dark-field image of a skin section from a mouse, whose hair follicles on the dorsal region are in early anagen, submitted to hybridization in situ using a sense probe of the EGR1 transcription factor (negative control). FIG. 4B is a photograph of the same histological section counter-stained with haematoxylin. This photograph (4B) serves as a reference for the dark-field image (4A).

FIG. 4C is a photograph of the dark-field image of a skin section from a mouse, whose hair follicles on the dorsal region are in early anagen, submitted to hybridization in situ using an antisense probe of the EGR1 transcription factor; the histological structures labeled radioactively by the probe are revealed by the accumulation of bright dots (silver grains). FIG. 4D is a photograph of the same histological section counter-stained with haematoxylin. This photograph (4D) serves as a reference for the dark-field image (4C). The labeled zones are indicated by arrows.

FIG. 5 shows the expression, by hybridization in situ, of the EGR1 transcription factor in the hair follicles in late anagen of the skin from the dorsal region of mice.

FIG. 5A is a photograph of the dark-field image of a skin section from a mouse, whose hair follicles on the dorsal region are in late anagen, submitted to hybridization in situ using a sense probe of the EGR1 transcription factor (negative control). FIG. 5B is a photograph of the same histological section counter-stained with haematoxylin. This photograph (5B) serves as a reference for the dark-field image (5A).

FIG. 5C is a photograph of the dark-field image of a skin section from a mouse, whose hair follicles on the dorsal region are in late anagen, submitted to hybridization in situ using an antisense probe of the EGR1 transcription factor, the histological structures labeled radioactively by the probe are revealed by the accumulation of bright dots (silver grains). FIG. 5D is a photograph of the same histological section counter-stained with haematoxylin. This photograph (5D) serves as a reference for the dark-field image (5C). The labeled zones are indicated by arrows. Analysis by hybridization in situ clearly shows that the transcripts are expressed specifically in the hair follicles in anagen.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES: EXPERIMENTAL DATA

### Example 1

### Expression of EGR1 at the Start of Anagen Induced by Ovariectomy, Analyzed using Affymetrix Chip Technology

Methods:

C57BL/6 female mice, 42 days old, whose hair follicles on the dorsal region were in telogen (Chase, 1954) were ovariectomized or not on Day 1 of the study. Ovariectomy performed during the telogen phase causes, in less than a week, massive transition of the hair follicles on the dorsal region to the anagen phase (Chanda, 2000) whereas the hair follicles on the dorsal region of the control animals are still in telogen.

Skin samples were taken from the dorsal region on Days 0, 1, 2, 4, 6 and 8 of the study. Part of the sample was used for confirming the transition to anagen by histological examination. The other part of the sample was used for performing an analysis of the transcriptome using Affymetrix chip technology.

Gene expression was analyzed on an Affymetrix station (microfluidic module; hybridization furnace; scanner; computer) following the supplier's recommendations. To summarize, the total RNAs isolated from the tissues are transcribed to cDNA. Starting from double-stranded cDNA, biotin-labeled cRNAs are synthesized using polymerase T7 and a biotin-conjugated NTP precursor. The cRNAs are then fragmented to small-sized fragments. All the stages of molecular biology are monitored using the “Lab on a chip” system from Agilent to confirm that the enzymatic reactions have been effective. The Affymetrix chip is hybridized with the biotinylated cRNA, rinsed and then fluorescence-labeled using a streptavidin-conjugated fluorophore. After several washings, the chip is scanned and the results are calculated using the MASS software supplied by Affymetrix. An expression value is obtained for each gene, as well as indication of the presence or absence of the value obtained. Calculation of the significance of expression is based on analysis of the signals that are obtained as a result of hybridization of the cRNA of a given gene with an oligonucleotide that hybridizes perfectly (“perfect match”) versus an oligonucleotide that contains a mutation (“single mismatch”) in the central region of the oligonucleotide.

Results:

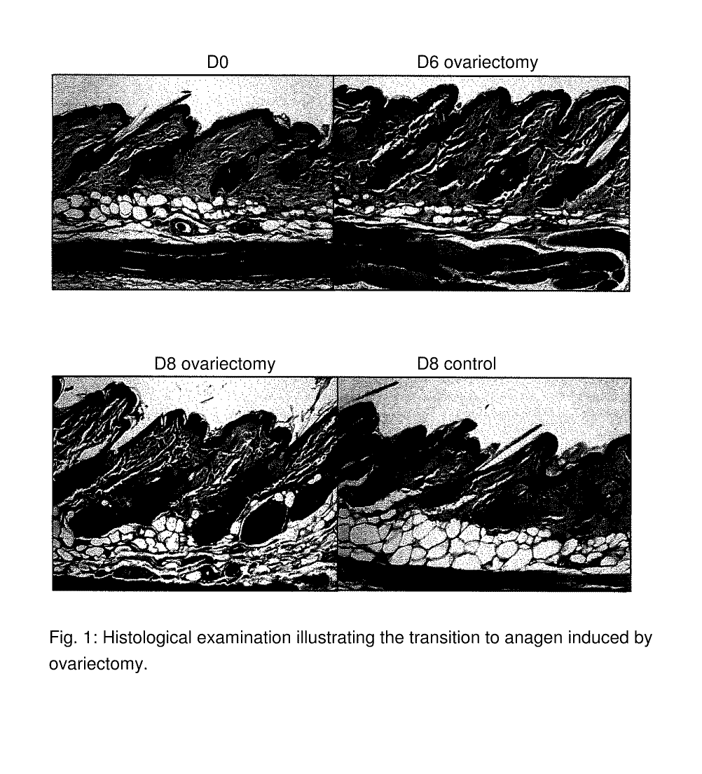

FIG. 1:

At the start of the study on Day 0, histological examination shows that the hair follicles on the dorsal region of the skin from the mice are in telogen phase (1A). In the ovariectomized mice, the hair follicles remain in telogen up to Day 6 of the study (1B). On Day 8 of the study, the hair follicles on the dorsal region of skin from all the ovariectomized mice are at the start of the anagen phase (1C). Conversely, the hair follicles on the dorsal region of skin from the control mice (not ovariectomized) remained in telogen. Thus, ovariectomy induced transition from the telogen phase to the anagen phase. The anagen phase was initiated after Day 6 of the study and was confirmed by histological examination on Day 8 of the study.

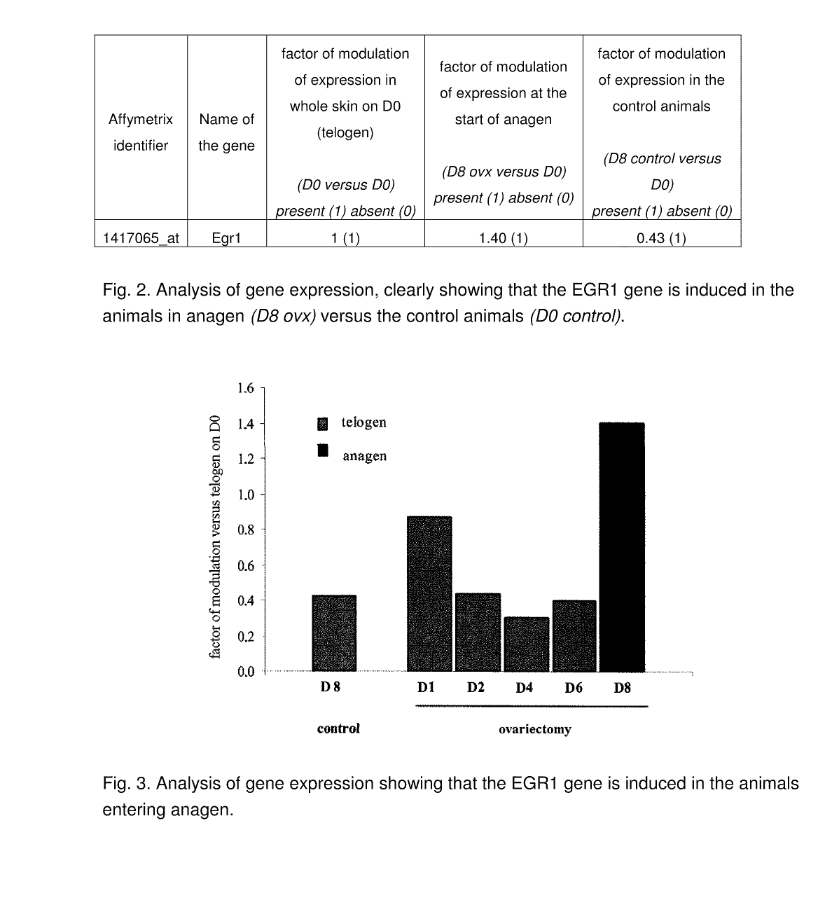

FIG. 2:

The EGR1 transcription factor is expressed in the telogen phase and in the anagen phase of the hair cycle. Differential analysis between expression in telogen on (D0) and anagen (D8 ovariectomized) shows that expression is stronger (factor 1.4) in early anagen relative to the telogen phase. In the control mice, expression of the EGR1 transcription factor is not induced, but is reduced relative to the start of the study.

FIG. 3:

The kinetics of expression of the EGR1 transcription factor at the start of the anagen phase following ovariectomy indicates that in the initial days following ovariectomy, expression of the EGR1 factor is reduced. Surprisingly, when the skin from the dorsal region of the ovariectomized mice shows the first morphological signs of the start of anagen (on Day 8 of the study), expression of the EGR1 factor is strongly induced relative to the preceding days. This induction correlates well with the start of the anagen phase, since expression of the EGR1 gene is not induced in the control animals whose hair follicles on the dorsal region remained in telogen.

### Example 2

### Expression of EGR1 in the Hair Follicles of Skin from the Dorsal Region of Mice by “Hybridization in Situ”

Methods:

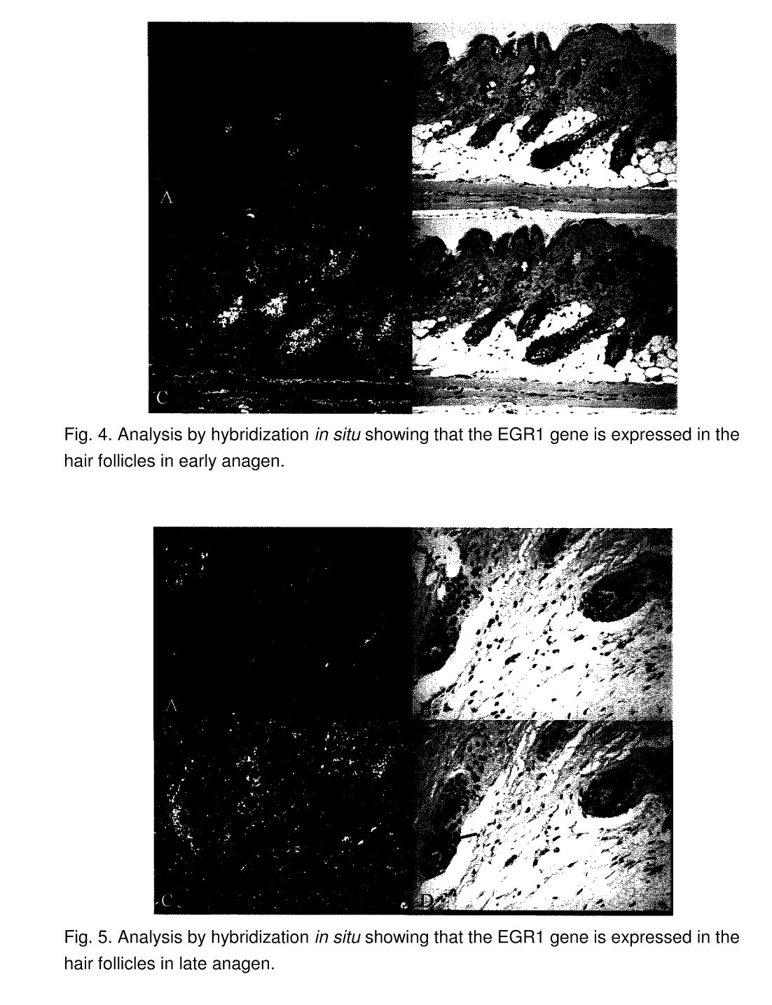

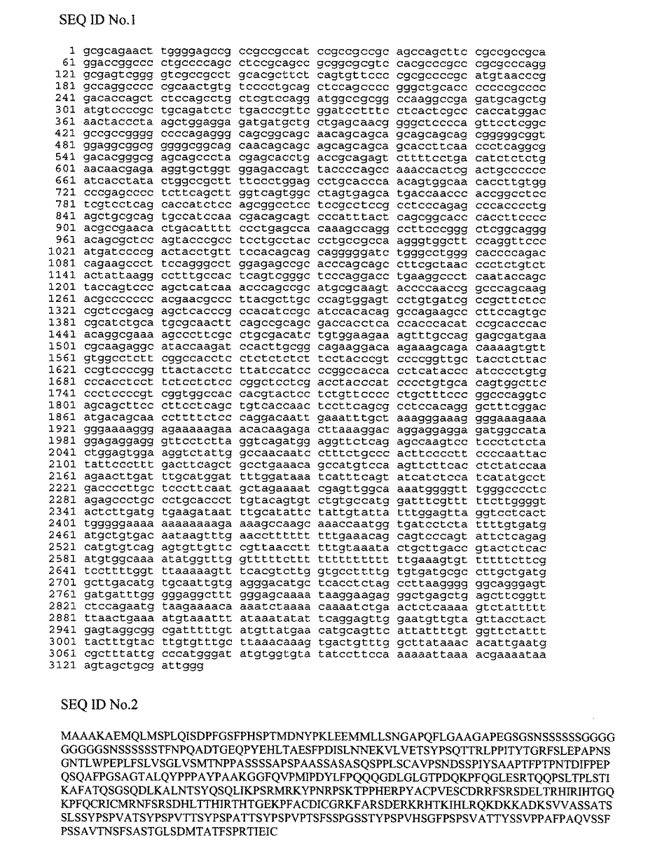

Sense and antisense probes were prepared from the EGR1 transcription factor by incubation of the linearized gene (2 μg) with 63 μCi of [³⁵S]UTP (1250 Ci/mmol; NEN, Massachusetts, USA) in the presence of RNA polymerase T7 or T3. Hybridization in situ was carried out on mouse tissue fixed with formaldehyde and embedded in paraffin. Sections (4 μm wide) were then deparaffined in toluene and rehydrated in an alcohol gradient. After drying, the various sections were incubated in a prehybridization buffer for two hours. Hybridization took place overnight in a hybridization buffer (prehybridization buffer with 10 mM DTT and 2×10⁶ cpm RNA/μl ³⁵S labeled) at 53° C. The excess of probe was removed and the sections were inclined in photographic emulsion LM1 (Amersham Biosciences, UK) and exposed in the dark at 4° C. for at least one month. The sections were then developed and counter-stained with haematoxylin and eosin. Following incubation in the presence of photographic emulsion, the histological structures labeled radioactively by the probe are revealed (accumulation of silver grains). A specific signal is manifested by positive labeling with the antisense probe (FIG. 4B and FIG. 5B) and the absence of labeling with the sense probe (FIG. 3A and FIG. 4A) used as negative control.

Results:

FIG. 4:

The images (A to C) show hair follicles from dorsal skin of mice at the very start of anagen. In FIG. 4A, there is no accumulation of silver grains (no labeling), which is in agreement with what the inventors expected as it corresponds to the negative control. FIG. 4C shows that the EGR1 transcription factor is expressed at the start of anagen and specifically in the epithelial part of the hair follicles. More particularly, the column of keratinocytes that forms in anagen II just above the dermal papilla is very strongly labeled (solid arrow) and the internal epithelial sheath that appears in anagen III is labeled (shaded arrow).

FIG. 5:

The images (A to C) show hair follicles from the dorsal skin of mice in mid-anagen. In FIG. 5A, it can be seen that there is no accumulation of silver grains (no labeling) which is in agreement with what the inventors expected as it corresponds to the negative control. FIG. 5C shows that the EGR1 transcription factor is expressed in mid-anagen and specifically in the epithelial part of the hair follicles. More precisely, the internal and external epithelial sheaths of the hair follicle in the middle of late anagen (IV-V) are strongly labeled.

### CONCLUSION

Example 1 shows that EGR1 is expressed in the skin and, surprisingly, is induced during transition between the telogen phase and the anagen phase. Example 2 confirms expression of EGR1 in the skin with hair follicles in early anagen and mid-anagen. It also shows that the EGR1 gene is, surprisingly, expressed specifically in the keratinocytes of the hair follicles in anagen.

All of these studies support the use of modulators of expression of the EGR1 factor in humans to achieve stimulation of growth of the hair follicles by inducing entry into the anagen phase. They also support the advantage of using EGR1 for diagnosis or prognosis of this pathology.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method of screening of candidate compounds for the preventive and/or curative treatment of alopecia, comprising the following stages:
a. preparing at least two biological samples or reaction mixtures;
b. contacting one of the samples or reaction mixtures with one or more of the compounds to be tested;
c. measuring the expression or of the activity of the Early Growth Response 1 protein, expression of its gene or of the activity of at least one of the promoters thereof, in the biological samples or reaction mixtures;
d. identifying and selecting the compounds for which modulation of the expression or activity of the Early Growth Response 1 protein, or modulation of the expression of its gene or modulation of the activity of at least one of the promoters thereof, is measured in the sample or the mixture treated in b) relative to the untreated sample or mixture.

2. The method as defined by claim 1, wherein the compounds selected in stage d) activate the expression or activity of the Early Growth Response 1 protein, or the expression of its gene or the activity of at least one of the promoters thereof.

3. The method as defined by claim 1, wherein the biological samples comprise cells transfected with a reporter gene bound operatively to all or part of the promoter of the gene coding for the Early Growth Response 1 transcription factor, and the stage c) comprises measuring the expression of said reporter gene.

4. The method as defined by claim 1, wherein the biological samples comprise cells expressing the gene coding for the Early Growth Response 1 transcription factor, and the stage c) comprises measuring the expression of said gene.

5. The method as defined by claim 3, wherein said cells are selected from among the keratinocytes and the fibroblasts of the dermal papilla or of the dermis.

6. The method as defined by claim 3, wherein said cells comprise cells transformed by a heterologous nucleic acid coding for the Early Growth Response 1 transcription factor.

7. The method as defined by claim 1, in which the expression of the gene is determined by measuring the rate of transcription of said gene.

8. The method as defined by claim 1, in which the expression of the gene is determined by measuring the rate of translation of said gene.

9. A medicament useful for the preventive and/or curative treatment of alopecia, comprising a modulator of the Early Growth Response 1 transcription factor.

10. The medicament as defined by claim 9, wherein said modulator comprises an activator of the Early Growth Response 1 transcription factor.

11. A regime or regimen for the aesthetic treatment of the scalp, comprising treating same with a modulator of the Early Growth Response 1 transcription factor.

12. An in vitro method of diagnosis or of monitoring the development of alopecia in a subject, comprising comparing the expression or activity of the Early Growth Response 1 protein, or of the expression of its gene or of the activity of at least one of the promoters thereof, in a subject's biological sample relative to a biological sample of a control subject.

13. The method as defined by claim 12, comprising determining the expression of said protein by assay of this protein by immunoassay.

14. The method as defined by claim 13, in which the immunoassay comprises an ELISA assay.

15. The method as defined by claim 12, comprising determining the expression of said gene by measurement of the amount of corresponding mRNA.

16. A regime or regimen for the treatment of alopecia, comprising administering to a subject in need of such treatment, for such period of time as required to elicit the desired response, a thus effective amount of a modulator of the Early Growth Response 1 transcription factor.

17. A regime or regimen for the treatment of alopecia, comprising administering to a subject in need of such treatment, for such period of time as required to elicit the desired response, a thus effective amount of an activator of the Early Growth Response 1 transcription factor.


SEQUENCE LISTING (2 sequences)

SEQ ID NO: 1 - DNA, 3136 bp, Homo sapiens
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 2 - protein, 543 aa, Homo sapiens
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC